(19) United States
(12) Patent Application Publication
(10) Pub. No.: US 2002/0052618 A1
(43) Pub. Date: May 2, 2002

(54) Analytical device with integrated lancet

(76) Inventors: Hans-Peter Haar, Wiesloch (DE); Hans List, Hesseneck-Kailbach (DE)

Correspondence Address: BRINKS HOFER GILSON & LIONE, ONE INDIANA SQUARE, SUITE 2425, INDIANAPOLIS, IN, 46204, US

(21) Appl. No.: 09/999,683

(22) Filed: Oct. 31, 2001

(30) Foreign Application Priority Data
Oct. 31, 2000 (DE) 100 53 930.0
Jan. 16, 2001 (DE) 101 01 658.1

(51) Int. Cl. A61B 17/32

(52) U.S. Cl. 606/181

(57) ABSTRACT

The invention describes an analytical device (1) which is suitable for collecting and examining body fluids and in particular blood. The analytical device (1) contains a test element (2) and a lancet (3) whereby the test element (2) contains the following components: 1.) a frame element (7), and 2.) at least one detection element (6) which can also be multilayered and can contain among others an erythrocyte separation layer, a spreading layer and an optical barrier layer which is directly or indirectly connected to the frame element (7) and the lancet (3) contains the following components: 1.) a needle (11) with a tip (23) and 2.) a lancet body (10) which at least partially surrounds the needle (11). The inventive device (1) is characterized in that the lancet body (10) is movably connected to the frame element (7) of the test element (2) i.e. it can be folded or swung out such that the lancet (3) can adopt a storage position and a lancing position, the needle (11) being aligned in the storage position essentially parallel to the plane of the test element (2) and aligned in the lancing position essentially orthogonal to the plane of the test element (2).

### BACKGROUND OF THE INVENTION

The present invention concerns analytical devices for collecting and examining body fluids, and in particular blood, containing a test element and a lancet wherein the test element contains a frame element and at least one detection element which is directly or indirectly connected to the frame element and the lancet contains a needle with a sharp tip and a lancet body which at least partially encloses the needle. The invention additionally concerns a system for storing analytical devices and a system for determining the presence or the content of an analyte in blood comprising a measuring instrument to measure and display the change of a characteristic property of a test element which correlates with the analyte and a magazine which is suitable for holding analytical devices.

So-called carrier-bound tests are used for the qualitative or quantitative analytical determination of components of body fluids and in particular of blood. In these carrier-bound tests the reagents are present on or in appropriate layers of a solid test carrier which is contacted with the sample. The reaction between the liquid sample and reagents leads to a detectable signal such as a color change or a change in current or potential. The detection signal can be evaluated visually or with the aid of an instrument; in the case of a color change usually by reflection photometry, in the case of a change in current or potential by an amp meter or volt meter, respectively.

Test carriers are often designed as test strips which are basically composed of an elongate carrier layer made of a plastic material and detection layers mounted thereon as test fields. However, test carriers are also known which are designed as quadratic or rectangular slides or in which the functional layers are held by a plastic frame. In the following, the general term “test elements” is used.

The determination of the content of certain analytes in blood such as glucose or lactate requires the collection of an adequate quantity of sample (blood) and the provision of a suitable measuring system for the analyte. In addition to doctor's practices and analytical laboratories, medical laymen are increasingly carrying out such determinations for their own use. Measuring systems that are intended to be used by the person to be examined are widespread especially for determining and monitoring the blood sugar value, i.e. blood glucose content in the case of diabetics, and also to determine other parameters such as the lactate content or cholesterol level.

Conventional measuring systems often contain test elements in the form of so-called test strips which, in conjunction with appropriate measuring instruments, allow the determination of one or several analytes in blood. In addition the user generally requires a lancet which is used to pierce the skin of certain body regions such as the finger pad or earlobe so as to obtain blood for the measurement. Various manufacturers offer so-called lancing devices for a comfortable collection of blood which drive lancets into the skin in a controlled and guided manner and hence control the puncture depth and minimise the pain.

Since several separate components are required in order to measure an analyte in blood (test elements, measuring instrument, lancing device, lancets etc.) which have to be carried with the user for analyses outside the home such as when travelling or for sport, it is understandable that especially diabetics, which also have to carry insulin and a syringe, would consider it desirable to reduce the number of individual components that have to be carried.

There has been no lack of different attempts to reduce the number of individual parts that are necessary to carry out a blood parameter determination. One solution described in the prior art is to combine the required components such as the measuring instrument, lancing device, lancets and test elements in a joint blood collection and measuring system whereby the lancet and test element in particular are combined to form a single analytical device. It is advantageous when this analytical device can be provided, used, evaluated and disposed off as a magazine and in an automated manner.

In the prior art there are basically two different approaches to analytical devices in which a flat test element is combined with a lancet in a single object (analytical device). On the one hand, concepts have been described in which the lancet or its needle or its spike executes a lancing movement that is essentially perpendicular to the plane of the test element (as described for example in U.S. Pat. No. 5,035,704 and FIGS. 2 and 3 of DE-A 198 55 443, DE-A 198 55 458 and DE-A 198 55 465). In the other approach, this lancing movement is such that the lancet or its needle or spike are moved essentially parallel to the plane of the test element (cf. FIG. 1 in DE-A 198 55 443, DE-A 198 55 458 and DE-A 198 55 465). A common feature of both variants is that the orientation of the lancet relative to the test element remains the same during the lancing movement (lancing position) as well as in the resting position (storage position).

A test element with an integrated lancet which can be stacked in magazines is described in U.S. Pat. No. 5,035,704. The test element is composed of a flat, rectangular (plastic) frame on the upper side of which the lancet is inserted and which carries a detection element on the underside parallel to the basal surface of the frame. The detection element only covers a part of the underside of the frame; the remaining part is practically open and serves as an opening through which the lancet tip can pass. A wicking material covering the whole surface of the detection element is located between the lancet and detection element and extends into the part of the basal surface not covered by the detection element. The wicking material is intended to transport blood from the puncture site of the lancet in the skin to the detection element. The lancet is composed of a metal sheet provided with punched holes which on the underside, i.e. the side facing the detection element, carries a central movable tongue, a pointed metal spike orientated essentially perpendicular to the metal sheet (and thus to the plane of the detection element). The tip of the spike is within the periphery of the test element frame in the resting state.

The test element from U.S. Pat. No. 5,035,704 is operated by placing the underside of the test element on the skin surface and the movable tongue with the spike is moved downwards with a plunger. In this process the spike pierces the skin surface through a hole in the wicking material and makes a small wound from which blood emerges after retracting the spike. The blood is transferred by the wicking material of the test element lying on the skin surface to the detection element and is analysed there.

DE-A 198 55 443, DE-A 198 55 458 and DE-A 198 55 465 describe, inter alia, various embodiments of analytical devices containing a test element and a lancet and are referred to therein as “test cassette”.

Flat, essentially rectangular “test cassettes” are disclosed in FIGS. 1 and 2 (and in the accompanying passages of the inventive description) of the three unexamined laid-open patent applications mentioned above in which a lancet element is inserted into a plastic frame consisting of several parts. The lancet element is composed of a lancet needle which is held by a plastic frame. The purpose of this plastic frame is to act as a guide element for the lancet needle and it is also used like a spring element to move the lancet needle back into the starting position after the lancing movement is completed. In contrast to the test element from U.S. Pat No. 5,035,704, the lancing movement of the lancet needle is essentially parallel to the plane of the detection element in the embodiments described in FIGS. 1A to 1E and 2A to 2B of DE-A 198 55 458. The detection element can be supplied with blood either via a capillary channel which either begins in the area of the exit port of the lancet needle or at any desired position of the test cassette housing.

FIG. 2C of DE-A 198 55 458 describes a similar test cassette in which the detection element is situated in a side surface of the plastic frame and surrounds the exit port of the lancet needle. In this embodiment the lancing movement of the lancet needle is essentially orthogonal to the plane of the detection element.

FIG. 3 of DE-A 198 55 443, DE-A 198 55 458 and DE-A 198 55 465 discloses elongate, cylindrical “test cassettes” in which the lancet is guided in a plastic sheath and the lancet needle protrudes from the basal surface of the “test cassette” when the lancing movement takes place. The exit port of the lancet needle can be surrounded by a detection element. As is also the case with the test element from U.S. Pat. No. 5,035,704, the lancing movement of the lancet needle in the embodiment described in FIG. 3 of DE-A 198 55 443, DE-A 198 55 458 and DE-A 198 55 465 is essentially perpendicular to the plane of the detection element. In this embodiment the lancet needle is surrounded by a lancet body which guides the lancet needle in conjunction with the cylindrical plastic sleeve of the “test cassette”. The lancet body and cylindrical plastic sleeve additionally interact with a spiral spring such that the lancet needle is returned back to the starting position after the lancing movement is completed.

The object of the present invention is to provide an analytical device containing a test element and a lancet which can be easily and safely stacked and wherein the lancet can be stored in such a manner that accidental injury can be largely excluded. In addition the analytical device should be characterized by small dimensions and be suitable for automated handling in an analyser.

The object is achieved by the subject matter of the invention.

### SUMMARY OF THE INVENTION

The invention concerns an analytical device which is suitable for collecting body fluids and in particular blood as well as for their examination immediately afterwards. The analytical device according to the invention contains a test element and a lancet. The test element comprises a frame element, at least one detection element which is directly or indirectly connected to the frame element and optionally other components such as a zone which is suitable for taking up excess sample liquid and a support for the detection element and/or for the zone that is suitable for taking up excess sample liquid. The lancet contains a needle comprising a tip and a lancet body which at least partially surrounds the needle. An essential feature of the analytical device according to the invention is that the lancet body is movably connected to the frame element of the test element, i.e. it is hinged or pivoted in such a manner that the lancet can adopt a storage position and a lancing position that is different from the storage position. In the storage position the needle is essentially parallel to the plane of the test element. In contrast it is essentially orientated orthogonal to the plane of the test element in the lancing position. In the lancing position the tip of the needle points essentially towards the test element.

An “analytical device” is understood in the sense of the invention as a device which is composed of at least one test element with an integrated lancet. The lancet is used to obtain a sample of a body fluid which is subsequently tested for the presence and/or the content of an analyte with the aid of the test element. Hence the analytical device according to the invention is suitable for obtaining a body fluid and in particular blood from a person to be examined. In this process the skin of this person is rapidly punctured by the lancet to a defined piercing depth resulting in a minute wound. A droplet of the body fluid and in particular blood of usually less than 1 μl to a maximum of 100 μl in volume collects on the surface of the wound. The body fluid is preferably used directly after collection for a diagnostic examination with the aid of the test element.

The analytical device according to the invention can be used in particular to obtain blood, preferably capillary blood from a body region such as a finger pad or an earlobe of an individual. The analytical device can be used by the individual to be examined himself, for example a diabetic who would like to determine his blood glucose content, or by another person, e.g. a doctor or nurse, to collect and examine blood samples.

The analytical device is preferably suitable for storage in a magazine and can be evaluated in a substantially automated process in a measuring instrument. For example analytical devices according to the invention can be stored in a magazine either stacked on top of one another or next to one another in the form of a chain.

The test element contains a shape-imparting stiff component which is referred to in the following as a frame element. The frame element serves to mechanically stabilize the detection element, to simplify the handling of the analytical device and ensures, in conjunction with the hinged lancet, that there is no risk of unintentional injury on the lancet needle.

In a particularly simple embodiment of the inventive test element, the frame element can be a stiff e.g. rectangular plastic foil or a stiff cardboard strip which carries the detection element on one side and on the other side is movably connected to the lancet, for example, by a film hinge or a strip of adhesive tape. The frame element may have a cut-out so that the lancet needle can optionally pass through the frame element during the lancing process.

However, the frame element is preferably an essentially rectangular, flat plastic formed piece e.g. an injection molded part. In addition to the frame itself, the frame element can also provide a support surface for the detection element in its interior. However, it is also possible that only the edges of the detection element are held by the frame. In both cases the detection element is permanently connected to the frame element.

Of course the frame element can also have several parts. The frame element can, for example, be composed of two halves and the detection element can be clamped between the two halves like the frame of a slide.

Corresponding test elements are known from the prior art for example from EP-A 0 885 591, EP-B 0 535 480 and EP-B 0 477 322.

Alternatively, the detection element can be attached to a support layer that is different from the frame element such as a transparent plastic foil which is in turn held by the frame element. This variant may be advantageous for the manufacture of the analytical device. In this case the detection element is indirectly connected to the frame element via the support layer.

The detection element is essentially composed of a detection layer containing reagent which is mounted on a support such as the bearing surface of the frame element described above or of a separate support layer. The detection layer contains the reagents that are required to detect the target analyte in the sample liquid to be examined. When the target analyte is present in the sample liquid, the reagents in the detection layer generate a signal, preferably a color change or current flow, that can be observed directly or indirectly by optical or electrical means. The detection layer can for example comprise a paper impregnated with analytical reagents and auxiliary substances or a plastic foil coated with reagents, fillers and film formers. It is also possible that the detection element is a membrane containing reagents or an electrode coated with reagents. Such detection elements that are suitable according to the invention for the analytical test element are known to a person skilled in the art in many different embodiments, for example from EP-A 0 821 234, EP-B 0 575 364, EP-A 0 016 387, EP-A 750 196, EP-A 0 654 659.

Analytical reagents in connection with the present invention basically means any type of detection reagents and other reactive auxiliary substances that are usually used in analytical and/or diagnostic test elements. These include but are not limited to indicators, mediators and labelling substances, buffer substances, spreading and wetting agents, activators, biochemical reagents, enzymes, proteins, peptides, antigens or antibodies and fragments thereof, happens and/or nucleic acids. Such reagents are known to a person skilled in the art for numerous analytical and/or diagnostic purposes. Even if reagents are often referred to in the following text this also includes according to the invention the possibility that only one reagent is used.

Detection elements that are suitable for the invention do not have to be composed of a single layer. Rather the detection element can be composed of two or several layers having different functions which are arranged horizontally or vertically next to one another. For example the detection element can be multilayered and among others contain an erythrocyte separation layer, a spreading layer and an optical barrier layer.

In a preferred embodiment the test element can contain several detection elements. These can for example be used to determine an analyte at different concentrations or they can be specific for different analytes.

Furthermore a detection layer can be specific for one or several target analytes. If it is specific for several analytes, the reagents for the various analytes can be accommodated in separate areas of the detection layer such that it is possible to unequivocally assign the detection results to a particular analyte. Technologies for manufacturing such detection layers having several separate areas are known to persons skilled in the art. Examples thereof are printing processes such as screen printing, ink-jet printing, photolithographic methods or simply the attachment of variously impregnated test papers to a common support.

In addition to the detection element, the test element can contain other components. For example, a zone for taking up excess sample liquid can be provided in the test element. Such a zone is preferably in direct proximity to the detection element. It can be in a liquid transfer-enabling contact with the detection element for example by placing them end-to-end, by slightly overlapping the connecting edges or by means of a connecting channel such that excess sample liquid can flow directly into the zone.

In a preferred embodiment the test element can contain a support layer for the detection element and optionally for other components. The support layer in this embodiment is preferably connected directly to the frame element such that the detection element is in turn connected indirectly to the frame element.

Suitable support layers are for example plastic foils and plastic formed parts, coated cardboards, glass, ceramics, metal sheets and the like. The support layer should be preferably inert towards the sample materials and reagents that are used, and not be attacked by them or react with them. For example, foils made of inert water-resistant plastics such as polyethylene, poly-propylene, polystyrene, polycarbonate, polyethylene terephthalate, polyamide and such like have proven to be suitable according to the invention.

In a preferred case using test elements that are to be evaluated optically, the support layer or the supporting surface of the frame element should be made to be transparent for optical measuring methods if the measurement is carried out from the side of the test element which rests on the support layer or the support surface of the frame element. The support layer and/or support surface can for example be composed of a transparent material or have an opening which allows an optical measurement. Such measures are familiar to persons skilled in the art.

The lancet contained according to the invention in the analytical device has a needle made of metal, ceramics or plastic one end of which (the point) has a pointed shape and is optionally ground sharp for example by means of a grinding process. At least the rear part of the lancet needle (the blunt end) facing away from this tip is completely or partially enclosed by a lancet head made of plastic. This is usually manufactured by positioning the lancet needle in a plastic jet mold and spraying on the lancet body. It is also possible that the lancet body is composed of several connected parts.

The lancet body serves to hold and guide the lancet needle and represents the connection between the lancet and test element. An essential feature of the analytical device according to the invention is that the lancet body is movably connected to the frame element of the test element, i.e. it is hinged or pivoted. The lancet body and the frame of the test element can be manufactured in a preferred embodiment in one piece as an injection molded part. In this case the lancet body and frame element are preferably connected by a film hinge. Alternatively the frame element and lancet body can be individual injection-molded parts movably connected together by means of a joint or hinge.

In this manner the lancet can adopt at least two defined orientations relative to the test element plane or to the plane of the test element. On the one hand, the lancet needle (and thus also the lancet) can lie parallel to the plane of the test element which is referred to in the following as the “storage position”. On the other hand, an orientation is possible in which the lancet needle lies essentially orthogonal i.e. perpendicular to the plane of the test element in which case the tip of the needle points towards the test element. The latter position of the lancet should be referred to as the “lancing position”. In addition, it is preferable that the detection element lies essentially parallel to the test element.

The analytical devices according to the invention can be stacked in a space-saving manner in the storage position. The parallel orientation of the lancet relative to the test element results in compact outside dimensions of the analytical device. The lancet can preferably be almost completely stowed away within the boundary surfaces in the frame element of the test element which will be described in more detail in the following figures. As a result the tip of the lancet needle is not exposed, and thus the risk of injury is minimized. In addition it enables a simple stacking of several analytical devices.

In the lancing position the lancet is swung out from the plane of the test element and is perpendicular to it. In this position the needle tip of the lancet needle is preferably at first concealed within the boundary surfaces of the frame element in order to almost completely prevent accidental injury on the needle tip. The end of the lancet which is surrounded by the lancet body protrudes from the boundary surfaces of the frame element in the lancing position and can be easily engaged by a drive element in this exposed position.

The lancing movement of the lancet is initiated by a drive element such as a hammer or plunger which is part of a corresponding measuring instrument or of a corresponding lancing device acting on the blunt end of the lancet needle or on the lancet body located at the blunt end of the lancet needle. The drive element can cause a forward movement of the lancet needle i.e. a movement towards the area of skin to be punctured as well as its return movement to the starting position. The drive element preferably acts linearly on the lancet which in turn executes a linear lancing movement.

As will be described in the following in more detail in conjunction with the figures, in a preferred embodiment the drive element firstly are tensions the return spring of the lancet. The lancet needle is only driven forward in a subsequent step, i.e. towards the skin surface to be pierced. As a result of the pretensioning of the return spring the lancing movement of the needle is completely controlled by the drive element of the measuring instrument or of the lancing device. Hence a guided controlled path-time course for the lancet needle is achieved during the lancing movement which results in a largely pain-free piercing.

During the lancing the lancet preferably penetrates the plane of the detection element. However, this does not necessarily mean that the lancet needle has to penetrate through the detection element, although this is possible but less preferred. In a preferred embodiment, the test element has a cut-out or opening in the area of the detection element through which the lancet needle can pass during the lancing process without touching the detection element. The detection element preferably surrounds this opening. Alternatively such a cut-out can be located in another area of the test element for example in the area of the frame element or in the area of other functional zones of the test element. It is also possible that the lancet needle is guided past the outside of the frame element of the test element during the lancing movement.

The detection element is preferably located directly adjacent to the opening for the lancet needle. This ensures that after puncturing the user of the analytical device according to the invention does not have to move the pierced body region such as the pierced finger into another position to apply the blood sample to the test element. In contrast the body region can remain at the same position for this purpose so that the blood sample that collects on the skin is applied almost automatically to the detection element.

Alternatively the beginning of a capillary transport path can be provided in the area of the opening which can be used to transport a blood sample to a detection element that is not located in the immediate vicinity of the opening. The transport path can for example be a capillary channel or an absorbent wick each of which is in a liquid transfer-enabling contact with the detection element(s).

In order to prevent blood, which has been obtained by lancing and has collected as a droplet on the skin surface, from passing through the opening to the side of the test element facing away from the skin during use of the analytical test element, the opening can be closed with a membrane made of an elastic plastic. The membrane is pierced like a septum by the lancet needle during the lancing process and reseals after the needle is retracted due to its elasticity.

The frame element and lancet body are preferably injection molded parts made of an injection moldable material and in particular a plastic that is suitable for injection molding. Such plastics are known to persons skilled in the art, e.g. polystyrene, polyamides, polyurethanes, cellulose ethers and cellulose esters, polyethylene, polymethacrylic acid ester and other thermoplastic materials, hardenable duroplasts and vulcanized elastomers made of rubber of silicone rubber and, although less preferred, foam plastics. In a preferred embodiment the frame element and lancet body are manufactured as one part and connected by a film hinge.

The lancet body can contain a return spring in order to return the lancet needle to its starting position after completion of the lancing movement. This is preferably connected directly to the lancet needle or to the part of the lancet body surrounding the lancet needle. The return spring can, for example, be a leaf spring or a spiral spring made of metal that is compressed during the lancing process and moves the lancet needle into its starting position when it springs back into its relaxed state. However, the return spring is preferably a deformable elastic part of the lancet body and is, for example, injection molded as one piece from the same plastic as the other parts of the lancet body. Of course it is also possible to use a different plastic for the return spring than for the rest of the lancet body. This is for example possible in two component injection molding manufacturing processes.

In a preferred embodiment of the analytical device according to the invention a guide element, for example, in the form of a guide sleeve or a guide channel in the test element, or in the form of a double-leaf spring in the lancet body, can be present in the lancet body and/or in the test element, and in particular in the frame element of the test element, in order to optimize the lancing movement of the lancet needle.

For use in extensively automated test systems it has proven to be advantageous for the test element, and in particular for the frame element of the test element, to have a guide profile on two opposing outer sides that engages in a complementary profile in the corresponding parts of a magazine or a test instrument. In this manner the movement of the analytical device can be guided and it is also possible to fix it in the measuring position. For example, the analytical device according to the invention can be moved from a storage position, for example in a device magazine, into a piercing and/or measuring position in a measuring instrument.

When the analytical device is transported from the storage position into the piercing or measuring position, the lancet is—preferably simultaneously—moved from the storage position into the piercing position. This can take place automatically without being acted upon externally for example by utilizing gravitational force causing the lancet to swing down and straighten out. However, it is preferable for the movement of the lancet from the storage into the piercing position to occur in a guided manner. For this purpose it has proven to be advantageous for the lancet body to have at least one and preferably a pair of opposing external cams that can engage in corresponding guide grooves in the measuring instrument and/or the magazine. The linear movement of the analytical device from the storage position into the measuring position is preferably converted into a suitable “folding down” of the lancet by curved guide grooves for the cams of the lancet body.

In a similar manner, further transport of the analytical device after completion of the analytical determination from the piercing or measuring position into the subsequent position (for the purpose of ejection or to be stored again in the magazine) causes the lancet to be returned again into the storage position. In this connection it is possible that the storage position before reaching the piercing or measuring position is identical to or different from the storage position afterwards. It is only important that the lancet is returned from an orientation that is orthogonal to the plane of the test element into an orientation that is parallel to the plane of the test element. This largely prevents a person from being accidentally injured by the lancet of the analytical device.

A further subject matter of the invention is a system for storing analytical devices. This system comprises a magazine or storage container for analytical devices and a store of analytical devices according to the invention. These analytical devices can be stored in the magazine essentially on top of one another in the form of a stack or essentially end to end or adjacent to one another in the form of a chain.

The magazine preferably has complementary guide profiles for the frame element of the preferred embodiment of the analytical device. This can ensure a safe and guided transport of the devices from the magazine.

In an alternative equally preferred embodiment, the magazine of the system according to the invention can have guide grooves for the cams of the lancet body of the analytical device that are contained in one of the preferred embodiments of the analytical device described above. As described above the guide grooves interact with the cams in order to move the lancet from the storage position into the lancing position when the device is transported into the measuring or piercing position.

Another subject matter of the invention is a system for determining the presence or the content of an analyte in blood. The system according to the invention comprises a measuring instrument to measure and display the change of a characteristic property of a test element, which correlates with the analyte, and a corresponding analytical device that can be used in the measuring instrument as described above. The system can additionally contain a magazine which is suitable for holding the analytical devices according to the invention.

The measuring instrument preferably has complementary guide profiles for the frame element of the preferred embodiment of the analytical device. This can ensure a safe and guided transport of the devices into the measuring position and lancing position and fix the analytical device in this position.

In an alternative equally preferred embodiment, the measuring instrument of the system according to the invention can have guide grooves for the cams of the lancet body of the analytical device that are contained in one of the preferred embodiments of the analytical device described above. As described above, the guide grooves interact with the cams in order to move the lancet from the storage position into the lancing position during transport of the device into the measuring or lancing position.

The system according to the invention preferably has a push or pull device in the measuring instrument or in the magazine, such as a motor or spring-driven plunger, for the analytical device, which moves the analytical device into the measuring or piercing position. In addition it may be preferable for the measuring instrument to contain a device for driving the lancet of the analytical device according to the invention, preferably a plunger to drive the lancet needle.

The inventive advantage of safe storage of the lancet needle in a storage position, and the exposed presentation of the lancet needle in a piercing position that is different from the storage position, can of course also be utilized by an object that does not contain a detection element. In the broadest sense such an object can be referred to as a hinged lancet. This is a further subject matter of the invention.

The hinged lancet differs from the analytical device described above in that it contains no detection element and thus it is not a complete test element in the sense of the invention. However, it contains the frame element according to the invention to which it is movably connected, e.g. hinged. The frame element, the connection between the lancet and the frame element, and the lancet per se are designed like the devices according to the invention (containing a test element and lancet). In particular the lancet of the hinged lancet can adopt a storage position and a piercing position which is different therefrom. The lancet can be essentially parallel to the plane of the frame element in the storage position and, in contrast, be aligned essentially perpendicular to the plane of the frame element in the piercing position.

A corresponding additional subject matter of the present invention is a system for storing the hinged lancet according to the invention. This system contains a magazine or storage container for hinged lancets and otherwise corresponds to the statements made above in relation to the storage system for analytical devices according to the invention.

The invention is elucidated in more detail by the following figures.

### BRIEF DESCRIPTION OF THE DRAWINGS

FIG. 1 shows a perspective top view (FIG. 1a), front view (FIG. 1b) and top view (FIG. 1c) of a preferred embodiment of the analytical device according to the invention.

FIG. 2 shows schematically in a sequence of four partial figures (FIGS. 2a to d) a side view of the interaction between the preferred embodiment of the analytical device from FIG. 1 and a part of a measuring instrument during transport of the analytical device into or from the measuring position.

FIG. 3 shows in a sequence of four detail views (FIGS. 3a to d) the interaction between the analytical device and measuring instrument during the piercing process in a sectional view through the preferred embodiment of the analytical device from FIG. 1.

FIG. 4 is a schematic sectional view of a preferred embodiment of the system according to the invention containing a measuring instrument, a magazine for analytical devices and the embodiment of the analytical device from FIG. 1 that is preferred according to the invention.

FIG. 5 shows a schematic perspective top view of the underside of a preferred embodiment of the analytical device according to the invention. The analytical device in this case essentially corresponds to that shown in FIG. 1.

FIGS. 6 to 9 show other alternative preferred embodiments of the analytical device according to the invention in a perspective top view of the underside.

FIG. 10 shows a sequence of 3 partial figures (FIGS. 10a to c) of a perspective view of the underside of the test element (top) or in a schematic side view (bottom) the interaction of a preferred embodiment of the analytical device from FIG. 5 with a part of a measuring instrument during transport of the analytical device into or from the measuring position.

FIG. 11 shows in a schematic perspective top view of the underside, a stack of analytical devices preferred according to the invention as they are for example present in the magazine of FIG. 4.

The numerals in the figures denote:

1 analytical device

2 test element

3 lancet

4 guide plate

5 lancet plunger

6 detection element

7 frame element

8 zone for taking up excess sample liquid (waste zone)

9 opening for the lancet needle

10 lancet body

11 lancet needle

12 cam

13 return spring

14 needle plunger

15 spring plunger

16 cam guide groove

17 guide groove for the frame element

18 hinge

19 transport direction of the analytical device

20 folding direction of the lancet

21 septum

22 opening for the lancet plunger

23 needle tip of the lancet needle

24 guide sleeve

25 measuring instrument

26 optics module

27 magazine

28 pressure plate

29 housing

30 ejector

31 ejection channel

32 cover

33 plunger for the analytical device

34 depression for finger

35 opening for plunger 33

36 opening for the analytical device 1

### DESCRIPTION OF PREFERRED EMBODIMENTS

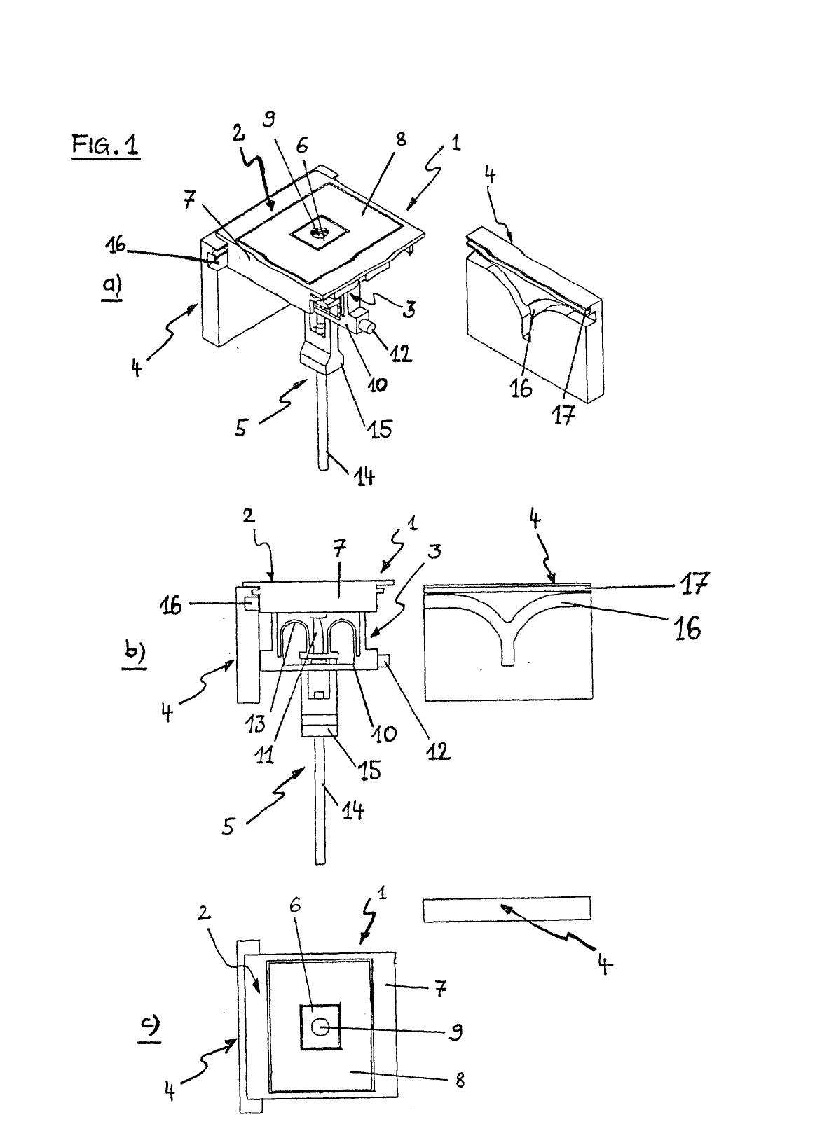

FIG. 1 shows a preferred embodiment of the analytical device (1) according to the invention. FIG. 1a shows a perspective top view of the analytical device (1) in conjunction with some components of a measuring instrument (guide plates (4), lancet plunger (5)). In order to better illustrate the guide plates (4), the right guide plate is shown in FIGS 1a (and in the following FIGS. 1b and 1c) folded out from its actual position to one side. The right guide plate (4) (that is swung out to one side) is, of course, mounted like the left guide plate (4) in the real embodiment of the analytical device (1) or of the associated measuring instrument.

The analytical device (1) is essentially composed of a test element (2) and a lancet (3). The test element (2) is composed of a frame element (7) which, in the preferred embodiment shown here, is an essentially rectangular, flat injection molded piece made of plastic. A rectangular detection element (6), which is surrounded by a zone of an absorbent material to take up excess sample liquid (waste zone (8)), rests centrally on the upper side of the frame element (7). The opening (9) for the lancet needle (11) of the lancet (3) runs perpendicularly to the plane of the detection element (6) through the frame element (7) and the detection element (6).

The frame element (7) has a profile on two opposing parallel outer edges that is used to guide the analytical device (1) by interacting with the guide groove (17) of the guide plate (4) during movement of the analytical device (1) into different positions of the measuring instrument.

The frame element (7) of the test element (2) is connected to the lancet (3) by means of a hinge, discussed below. The lancet (3) (as can be seen especially in FIG. 1b) is composed of a lancet body (10) which is also, like frame element (7), a plastic molded piece which is injection molded, and a lancet needle (11), which is preferably made of metal and has a ground point. The lancet body (10) has a pair of guide cams (12) on its side edges which engage in corresponding guide grooves (16) of the guide plates (4). The interaction between the guide grooves (16) and guide cams (12) during the movement of the analytical device (1) in the measuring instrument causes the lancet (3) to be swung down from the storage position into the piercing position.

The lancet body (10) also contains return springs (13) which are used to return the lancet needle (11) into the starting position after a lancing movement is completed.

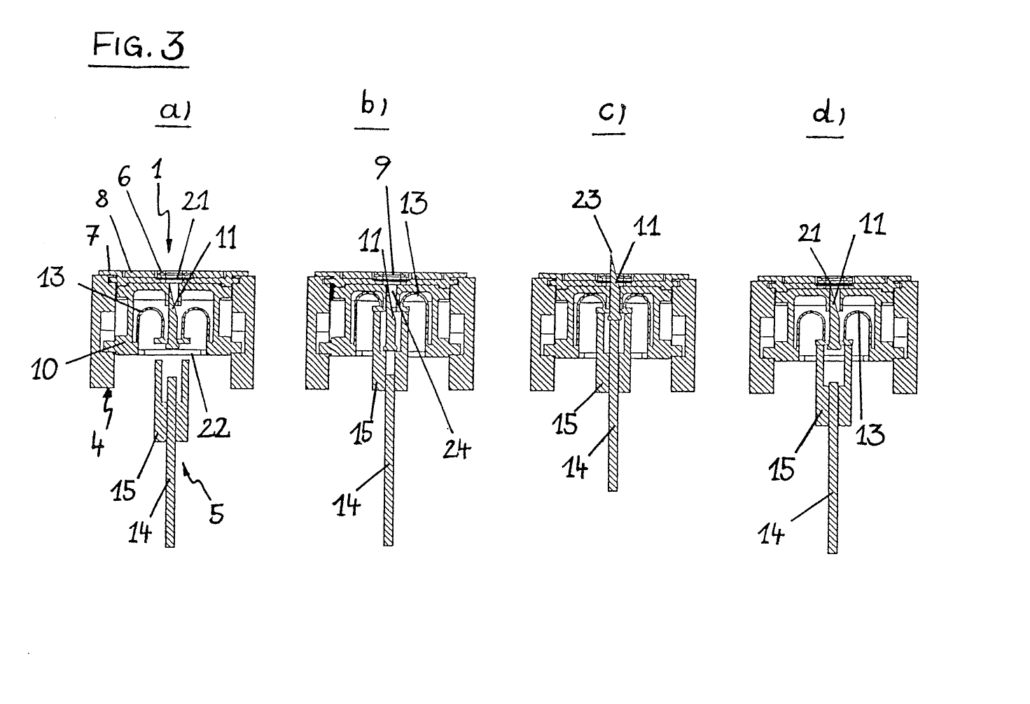

FIG. 1b also clearly shows how the lancet plunger (5) is configured to act on the lancet (3). The lancet plunger (5) contains a needle plunger (14) which interacts specifically with the lancet needle (11) and a spring plunger (15) which is designed to pretension the lancet return spring (13). The interaction of the individual plunger components with the lancet (3) is described in more detail in conjunction with FIG. 3.

FIG. 1c shows a top view of the analytical device (1). In particular this view again illustrates in which order the individual components of the test element (2) are arranged. A central cut-out in the test element (2) serves as an opening (9) for the lancet needle. The opening (9) is surrounded by an essentially rectangular detection element (6) that in turn is directly surrounded by a waste zone (8), which can for example be composed of a piece of absorbent paper. The detection element (6) and waste zone (8) are attached to a support surface of a frame element (7), for example, by gluing.

The surface of the analytical device (1) shown in the top view of FIG. 1c is the surface which comes into contact with the skin surface of the individual to be examined during operation of the analytical device (1). The individual to be examined, for example, places a finger on the upper surface of the analytical device (1). The dimensions of the analytical device (1) are preferably selected such that the surface of the detection element (6) and of the waste zone (8) can be covered by the finger of the individual to be examined. When the lancet (3) carries out the piercing operation, which is driven by the lancet plunger (5), the lancet needle (11) passes through the opening (9) and penetrates into the skin of the individual to be examined. After the lancet needle (11) is retracted, the pierced skin region of the individual to be examined remains in an unchanged position relative to the analytical device (1). The wound generated by the lancet needle (11) causes a blood droplet to form on the skin surface which is taken up by the detection element (6). Excess blood that may be present is taken up by the waste zone (8). The geometric arrangement of the opening (9) and detection element (6) enables the blood sample to be applied to the detection element (6) practically at the moment it is formed.

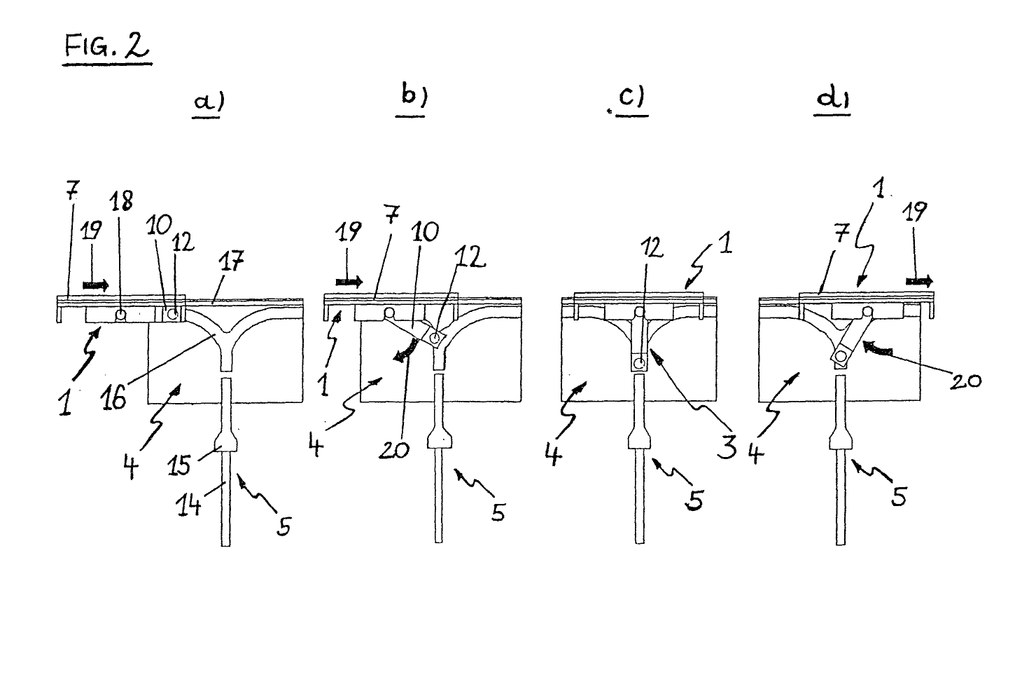

In FIG. 2 four partial figures in side view show how the analytical device (1) from FIG. 1 interacts with the guide plates (4) during transport of the analytical device (1) from a storage position into a measuring or piercing position and subsequently from this measuring or piercing position.

FIG. 2a shows the beginning of the transport of the analytical device (1) from the storage position into the measuring position. The storage position in which the lancet (3) is essentially completely within the boundary surfaces of the test element (2) is characterized by the lancet needle (11) being essentially parallel to the plane of the test element (2) and in particular to the plane of the detection element (6). This position can be seen in FIG. 2a. The measuring position corresponds to the state that is adopted by the analytical device (1) when the guide cams (12) of the lancet (3) have reached the vertical section of the guide grooves (16). In this state the lancet (3) is disposed exactly opposite to the lancet plunger (5) (composed of the needle plunger (14) and spring plunger (15)) (cf. also FIG. 2c). In this case the lancet needle (11) is aligned essentially perpendicular to the plane of the test element (2), and in particular to the plane of the detection element (6), and the needle tip points towards the test element (2).

As shown in detail in FIGS. 2a to 2d, the frame element (7) of the test element (2) is guided by the guide grooves (17) when the analytical device (1) is transported in direction (19). The transport direction (19) runs in a straight horizontal line through the guide plates (4). The curved guide grooves (16) interact with the cams (12) to ensure that during the linear movement of the analytical device (1) the lancet body (10) is swung around the hinge (18) in direction (20), i.e. essentially downwards until it reaches the measuring or piercing position which is shown in FIG. 2c. The analytical device (1) remains in this state until the lancing or measuring process is concluded. During this the lancet (3) is in the lancing position which differs from the storage position of the lancet (3) shown in FIG. 2a in that the alignment of the lancet needle (11) is essentially orthogonal to the plane of the test element (2).

After the measurement has been carried out, the analytical device (1) according to the invention is transported in direction (19) as shown in FIG. 2d. The curve in the guide grooves (16) ensure that the lancet (3) swings in direction (20) to a second storage position. The lancet needle (11) is again aligned parallel to the plane of the test element (2) in this second storage position in the final state.

FIG. 3 shows schematically in four detail views (FIGS. 3a-3d) the interaction of the lancet plunger (5) with the lancet (3) of the analytical device (1) from FIGS. 1 and 2. FIG. 3a shows the analytical device (1) in the measuring and lancing position which essentially corresponds to FIG. 2c. In the starting position shown in FIG. 3a the needle plunger (14) and spring plunger (15) of the lancet plunger (5) are arranged below the lancet (3). Lancet (3) is still in a resting position of the lancing position. The lancet needle (11) is held in the resting position by the return spring (13). The lancet body (10) fixes the position of the lancet (3) by interaction with the guide grooves (16) in guide plates (4) and thus ensures the lancet needle (11) has a well-defined alignment relative to the lancet plunger (5), relative to the test element (2), and in particular, relative to the detection element (6). FIG. 3a clearly shows that the test element (2), which is composed of the frame element (7) and the layered components i.e. detection element (6) and waste zone (8) that are attached to the support surface of the frame element (7), has a rubber membrane as a septum (21) in the area of the opening (9). The septum (21) can be pierced by the lancet needle (11) in the lancing process and can be tightly resealed again after the lancet needle (11) is pulled back into the starting or resting position.

As also shown in FIGS. 3a to 3d the lancet body (10) has an opening (22) for the lancet plunger (5). The opening (22) is situated on the side facing the blunt end of the lancet needle (11). The sharp end or tip (23) of the lancet needle (11) is surrounded in the resting state by a guide sleeve (24), which is also part of the lancet body (10). In addition, the guide sleeve (24) serves to enclose the tip (23) to avoid accidental injury on the lancet needle (11).

FIG. 3b shows how at the beginning of the lancing process the spring plunger (15) acts on a part of the lancet body (10) holding the lancet needle (11) and thus moves the return spring (13) into a tensioned state. FIG. 3c shows how, after the return spring (13) has been pretensioned by the spring plunger (15), the needle plunger (14) acts on the blunt end of the lancet needle (11) and pushes the tip (23) through the septum (21) such that the tip (23) of the lancet needle (11) protrudes from the surface of the analytical device (1) to penetrate through the skin of an individual to be examined. This piercing movement of the lancet needle (11) is guided by the guide sleeve (24) of the lancet body (10) and by the opening (9) of the test element (2).

FIG. 3d shows schematically how, after the lancet plunger (5), i.e. the needle plunger (14) and the spring plunger (15), has been pulled back, the return spring (13) moves the lancet needle (11) back into the starting position. In this process the septum (21) closes. The tip (23) of the lancet needle (11) is now again completely within the lancet body (10). The resealed septum (21) ensures that blood, which has collected on the upper surface of the test element (2) after the lancing process, cannot pass through to the underside of the test element (2) where it might potentially contaminate parts of the measuring instrument.

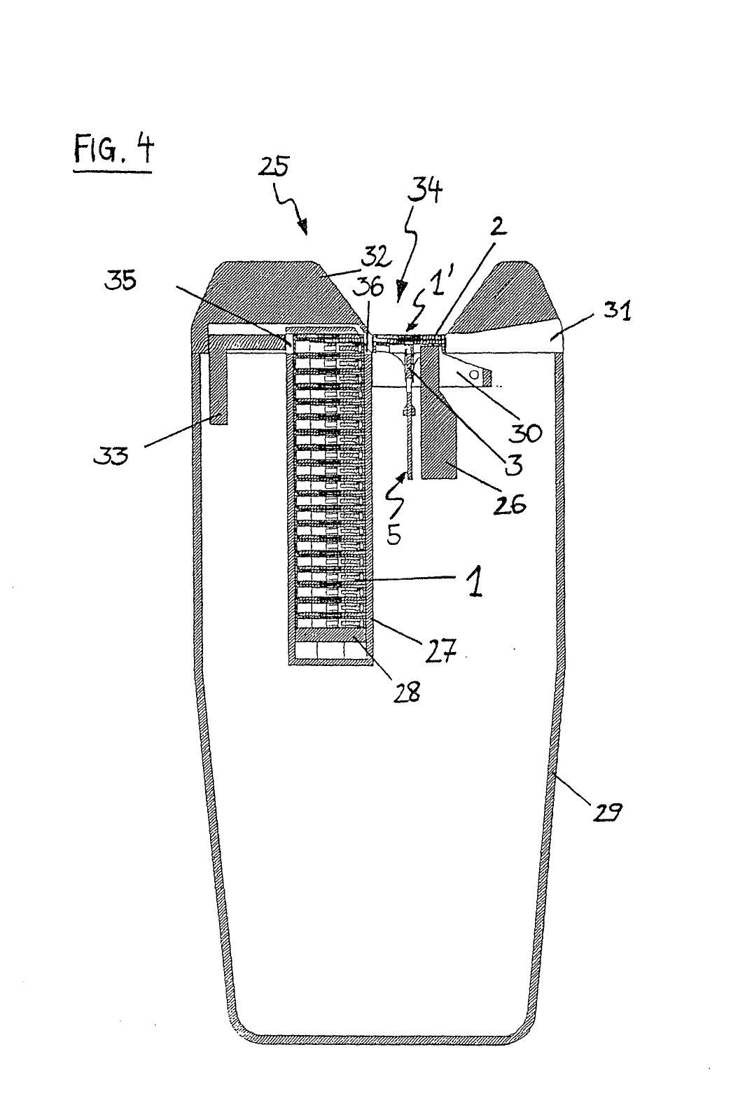

FIG. 4 shows a longitudinal section through a preferred embodiment of a measuring instrument (25). The measuring instrument (25) contains a magazine (27) for storing analytical devices (1) like those described in particularly preferred embodiments in conjunction with FIGS. 1 to 3. If required the magazine (27) can be removed from the measuring instrument (25) by removing the cover (32) and replaced by a new magazine. For this purpose the cover (32) is preferably attached by means of a hinge, not shown, to the measuring instrument housing (29).

The measuring instrument (25) additionally contains a plunger (33) that is used to remove an analytical device (1) from the magazine (27) into a measuring or piercing position. For this purpose the magazine (27) has an opening (35) through which the plunger (33) can pass and has an opening (36) opposite to opening (35) through which an analytical device (1) can pass from the magazine (27) into the measuring position. Lower analytical devices (1) can be moved upward to the position of the removed analytical device (1) in the magazine (27) by a pressure plate (28) which is either part of the magazine (27) or an integral part of the measuring instrument (25). The pressure plate (28) can be driven by a manually operated slide, a motor or a spring, not shown.

FIG. 4 shows an analytical device (1′) in the measuring or lancing position. The lancet (3) is swung out such that it is essentially perpendicular to the plane of the test element (2). The lancet plunger (5) which faces the lancet (3) is in the measuring or lancing position. In order to evaluate the detection element, the measuring instrument (25) has a movable optical module (26) which in the preferred embodiment can be lowered during movement of the analytical device (1). The test element is measured by the optical module (26) by known methods, for example, by reflection photometry.

The measuring instrument (25) additionally contains an ejecting device (30) which, in conjunction with the frame element (7) of the analytical device (1), removes the analytical device from the measuring instrument (25) via the ejection channel (31) after the measurement is completed. Alternatively, the used analytical device (1) can be transferred to another magazine (not shown) for storage and disposal.

The measuring instrument (25) has a depression or channel (34) in which, for example, a finger can be placed of an individual to be examined. The finger can be contacted with the analytical device (1′) in this depression (34). The finger can remain in an unchanged position during the entire lancing and measuring process.

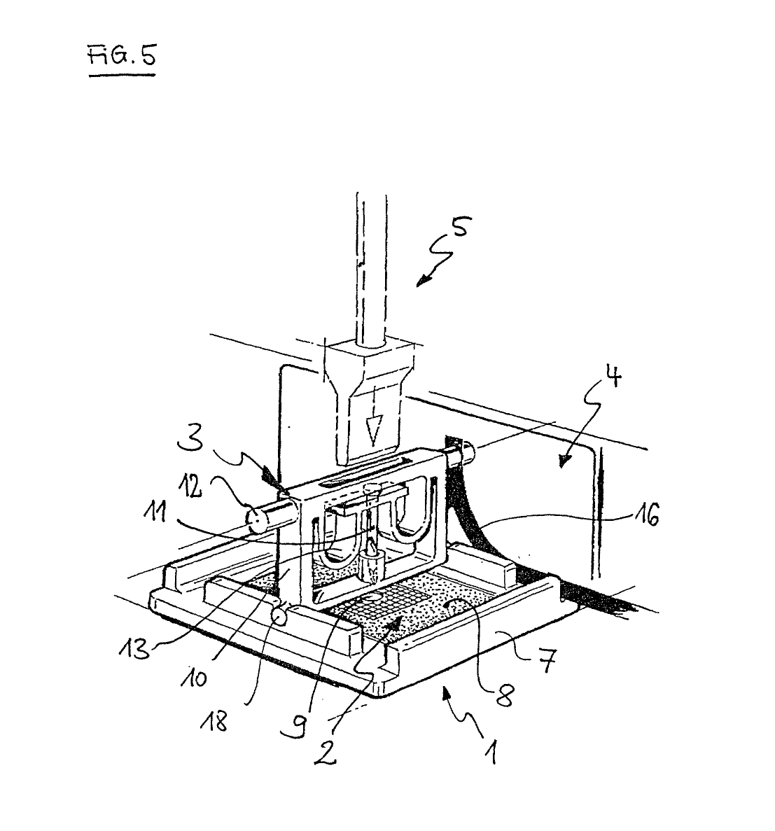

FIG. 5 shows a further preferred embodiment of the analytical device (1) according to the invention. The analytical device (1) of FIG. 5 corresponds essentially to the analytical device (1) shown in FIG. 1. The analytical device (1) contains a lancet (3) which is movably connected via a hinge (18) to the frame (7) of the test element (2). FIG. 5 also shows schematically the lancet plunger (5) which interacts during a piercing movement with the lancet (3) of the analytical device (1). The function and notation of the other elements of the analytical device (1) of FIG. 5 are essentially identical to those of the analytical device (1) from FIG. 1. Reference is explicitly made here to FIG. 1.

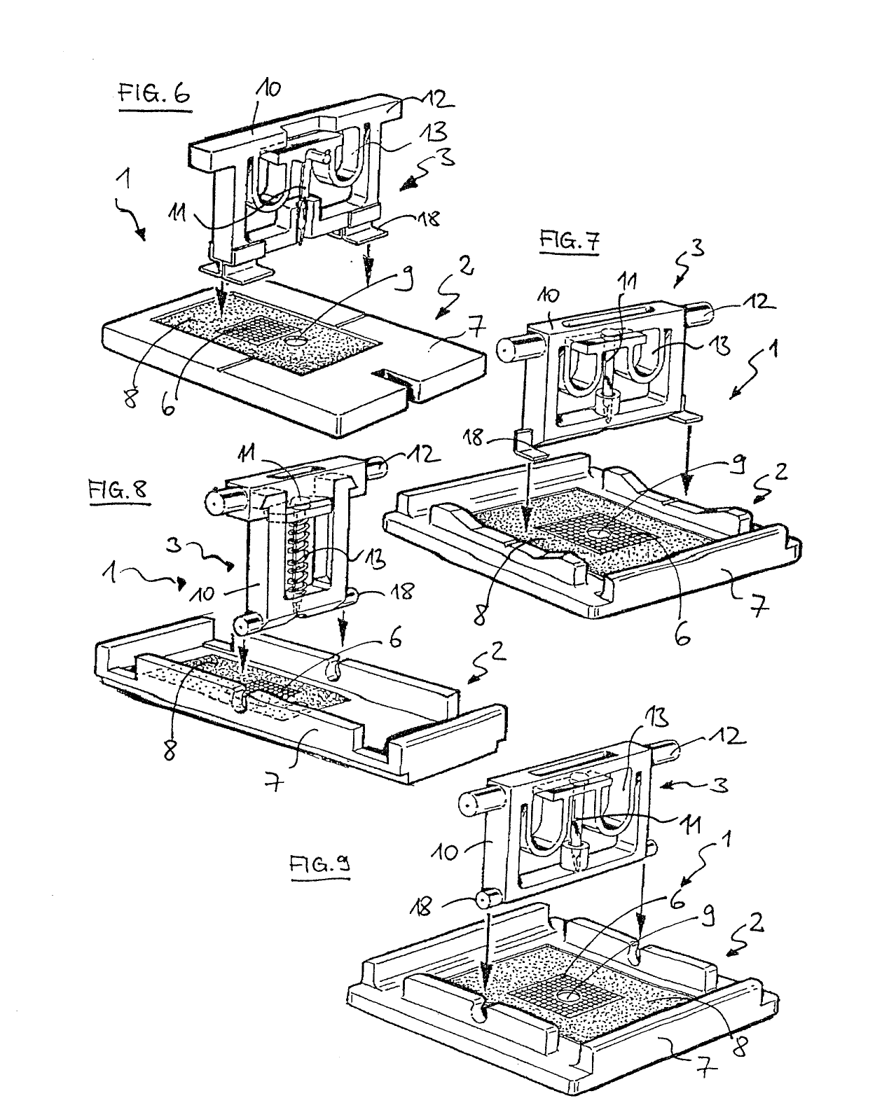

FIGS. 6 to 9 show alternative equally preferred embodiments of the inventive analytical device (1) in an enlarged fragmentary view. The analytical devices (1) of FIGS. 6 to 9 each contain a lancet (3) that is connected to a test element (2) via a hinge or joint (18). In FIGS. 6 to 9, the lancets (3) and the test elements (2) are, in each case, shown separated from one another. The arrows in these Figures indicate how the two elements (lancet (3) and test element (2)) can, for example, be assembled during manufacture. The analytical devices (1) of FIGS. 6 to 9 essentially correspond to the analytical devices (1) that have already been described in FIGS. 1 to 5. Explicit reference is made here to the description of these figures.

The analytical device (1) of FIG. 6 is essentially composed of foil or tape-like materials. In particular the frame (7) of the test element (2) and the lancet body (10) of the lancet (3) are made of foil material. The hinge (18) is also manufactured from a foil material. The lancet (3) and test element (2) are connected together via the foil joint (18). The connection can, for example, be made by gluing or welding.

FIGS. 7 to 9 show analytical devices (1) in which the frame (7) of the test element (2) and the lancet body (10) of the lancet (3) are composed of injection molded parts. In the analytical devices (1) of FIGS. 7 to 9, the lancet (3) and the test element (2) can be connected together via a foil hinge (18) as shown in FIG. 7 or by a joint (18) as shown in FIGS. 8 and 9, which is essentially composed of a pair of cylindrical pins that engage in corresponding recesses in the frame (7) of the test element (2).

As can be seen by comparing FIGS. 6, 7 and 9 with FIG. 8, the return spring (13) of the lancet (3) can have different designs. Whereas the return spring (13) in FIGS. 6, 7 and 9 is formed in one piece with the lancet body (10), a spiral spring (13) which is preferably manufactured from metal is shown in FIG. 8.

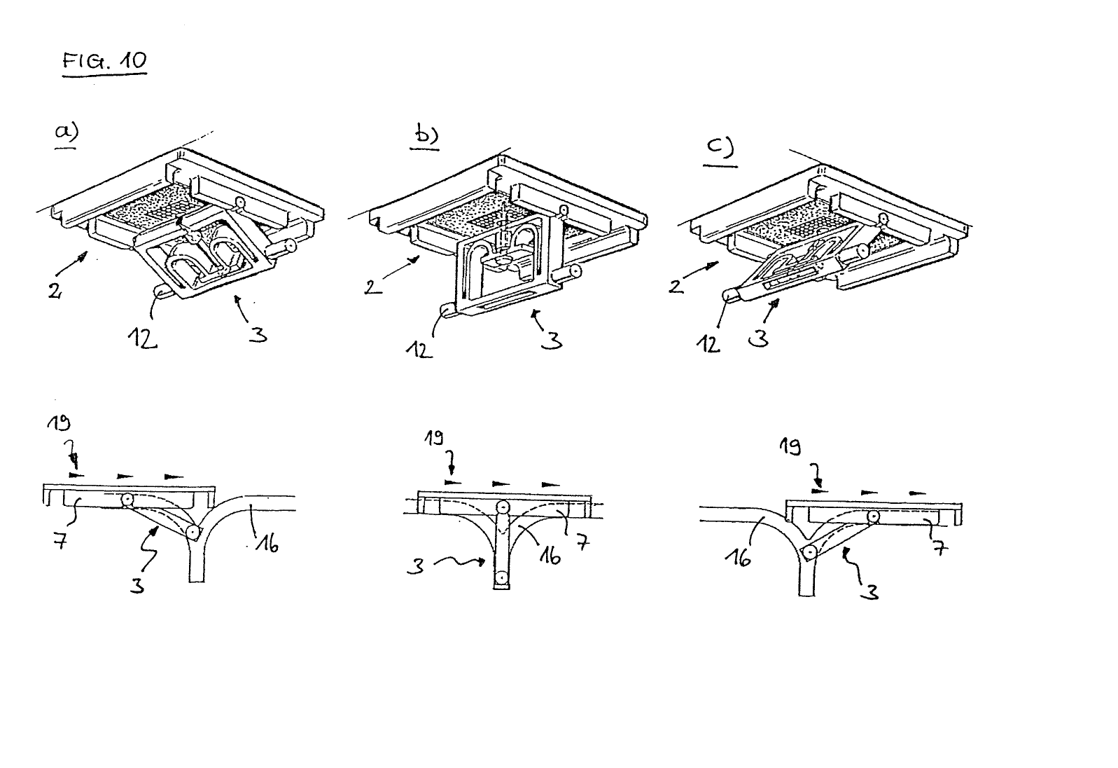

Three snap shots (FIGS. 10a, b and c) of the movement of the analytical device (1) during transport from the storage position into or out of the measuring position are shown in the perspective diagram of FIG. 10. The upper figure is in each case a perspective top view of the underside of the analytical device whereas the lower figure shows a simplified side view. The stages shown in FIGS. 10a to c correspond to those shown in FIGS. 2b to d. Reference is made to the description of FIG. 2, above, for details.

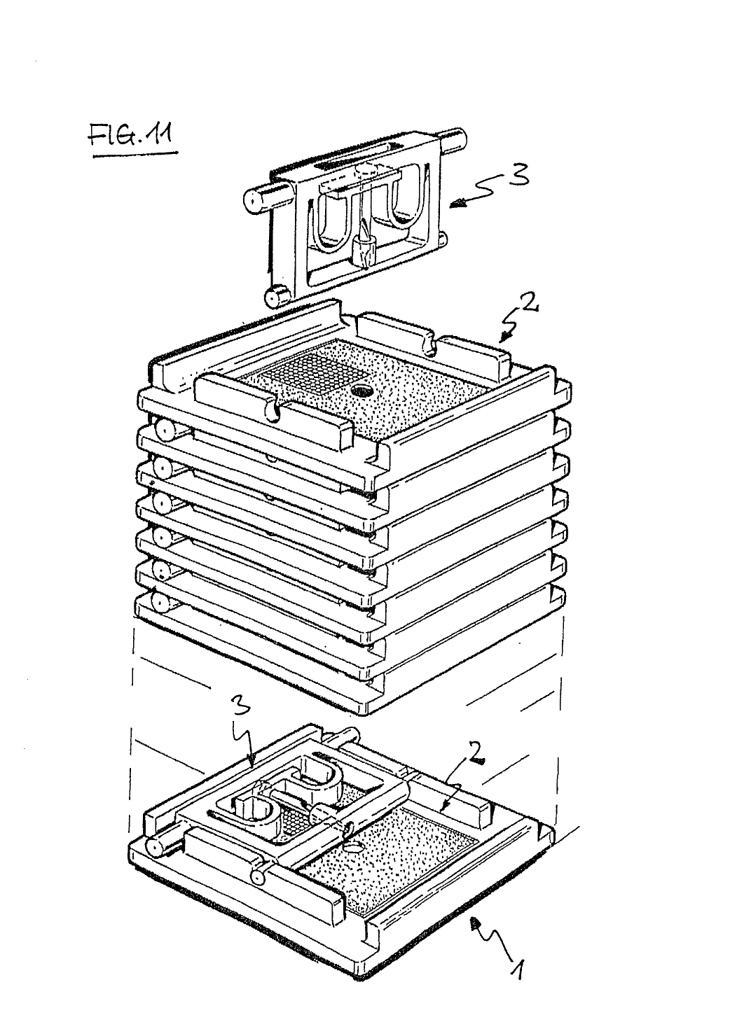

FIG. 11 shows schematically a perspective top view of the underside of a stack of analytical devices (1) as described in more detail in FIG. 5. Such a stack of analytical devices (1) is, for example, present in the magazine (27) of the measuring instrument (25) of FIG. 4.

The lowest analytical device (1) in FIG. 11 is in the storage position. In this position the lancet (3) is folded down into the frame (7) of the test element (2). The lancet needle (11) is essentially parallel to the plane of the test element (2).

In the uppermost analytical device (1) in FIG. 11, the lancet (3) and the test element (2) are again shown separately as in FIGS. 6-9. Of course, in reality the lancet (3) and the test element (2) are connected together via the hinge (18). In contrast to the lowest analytical device (1) of FIG. 11, the lancet (3) in the uppermost analytical device (1) in FIG. 11 is aligned essentially perpendicular to the plane of the test element (2). This would correspond to the alignment of these two components of the analytical device (1) in the lancing position.

1. An analytical device for collecting and examining a body fluid comprising: a test element including a frame element defining a plane and a detection element connected to the frame element, and a lancet including a needle having a tip and a lancet body at least partially surrounding the needle, the lancet body being movably connected to the frame element such that the lancet can adopt a storage position wherein the needle is aligned essentially parallel to the plane of the test element and a lancing position wherein the needle is aligned essentially orthogonal to the plane of the test element.

2. The analytical device as claimed in claim 1, wherein the test element contains a separate zone of absorbent material, which is suitable for taking up excess sample liquid, in the immediate vicinity of the detection element.

3. The analytical device as claimed in claim 2, further comprising a common support layer supporting the detection element and the zone of absorbent material.

4. The analytical device as claimed in claim 1, further comprising a hinge connecting the lancet body to the frame element.

5. The analytical device as claimed claim 1, wherein the frame element and the lancet body are injection molded.

6. The analytical device as claimed in claim 1, wherein the test element includes an opening for receiving the lancet needle.

7. The analytical device as claimed in claim 6, wherein the opening is in the area of the detection element.

8. The analytical device as claimed in claim 1 further comprising a return spring situated in the lancet body for the lancet needle.

9. The analytical device as claimed in claim 1 further comprising a guide sleeve for the lancet needle in the lancet body.

10. The analytical device as claimed in claim 1, further comprising guide plates containing guide profiles on two opposing parallel sides of the frame element.

11. The analytical device as claimed in claim 1, wherein the lancet body includes a pair of external cams.

12. A system for storing analytical devices comprising a magazine and a store of analytical devices, each analytical device having a test element including a frame element defining a plane and a detection element connected to the frame element, and a lancet including a needle having a tip and a lancet body at least partially surrounding the needle, the lancet body being movably connected to the frame element such that the lancet can adopt a storage position wherein the needle is aligned essentially parallel to the plane of the test element and a lancing position wherein the needle is aligned essentially orthogonal to the plane of the test element.

13. The system as claimed in claim 12, wherein the magazine contains complementary guide profiles for receiving the frame element of one of the analytical devices.

14. The system as claimed in claim 12, wherein the lancet body includes cams for causing movement of the lancet body relative to the frame element, and the magazine contains guide grooves for engaging the cams of the lancet body of one of the analytical devices.

15. A system for determining the presence or the content of an analyte in blood comprising: a measuring instrument for measuring a change of a characteristic property of a test element which correlates with the analyte and a magazine suitable for holding a plurality of analytical devices, each analytical device having a test element including a frame element defining a plane and a detection element connected to the frame element, and a lancet including a needle having a tip and a lancet body at least partially surrounding the needle, the lancet body being movably connected to the frame element such that the lancet can adopt a storage position wherein the needle is aligned essentially parallel to the plane of the test element and a lancing position wherein the needle is aligned essentially orthogonal to the plane of the test element.

16. The system as claimed in claim 15, wherein the measuring instrument contains complementary parallel guide profiles for guiding the frame element of the analytical device during movement relative to the measuring instrument.

17. The system as claimed in claim 15, wherein the lancet body includes cams for causing movement of the lancet body relative to the frame element, and the measuring instrument contains guide grooves for receiving the cams of the lancet body.

18. The system as claimed in claim 15, wherein the measuring instrument comprises a lancet plunger for driving the lancet of the analytical device when the lancet is in said lancing position.

19. A test element comprising: a frame element defining a plane and a lancet, the lancet containing a needle with a tip and a lancet body at least partially surrounding the lancet needle, the lancet body being movably connected to the frame element such that the lancet can adopt a storage position wherein the lancet needle is aligned essentially parallel to the plane of the frame element and a lancing position wherein the lancet needle is aligned essentially orthogonal to the plane of the frame element.

20. The test element as claimed in claim 19 wherein the frame element includes a detection element, an opening in the area of the detection element for receiving the lancet needle, a separate zone of absorbent material, which is suitable for taking up excess sample liquid, in the immediate vicinity of the detection element, and a common support layer supporting the detection element and the zone of absorbent material.

21. The test element as claimed in claim 20 wherein the lancet body includes a guide sleeve for guiding movement of the lancet needle relative to the lancet body, and a return spring for returning the lancet needle to an initial position from any displaced position.

22. The test element as claimed in claim 21, further comprising a hinge connecting the lancet body to the frame element, the lancet body including a pair of external cams for causing movement of the lancet body relative to the frame element.
